(19) United States
(12) Patent Application Publication
(10) Pub. No.: US 2013/0137168 A1
(43) Pub. Date: May 30, 2013

(54) BIOCHIP

(71) Applicants: Jeong Suong YANG, Yongin (KR); Sang Jin Kim, Hwaseong (KR)

(72) Inventors: Jeong Suong YANG, Yongin (KR); Sang Jin Kim, Hwaseong (KR)

(73) Assignee: SAMSUNG ELECTRO-MECHANICS CO., LTD.

(21) Appl. No.: 13/407,208

(22) Filed: Feb. 28, 2012

(30) Foreign Application Priority Data
Nov. 29, 2011 (KR) 10-2011-0126048

(51) Int. Cl. C12M 1/00

(52) U.S. Cl. 435/289.1

(57) ABSTRACT

There is provided a biochip including a first substrate having a plurality of biological materials disposed thereon at predetermined intervals; first coupling units respectively extending from both ends of the first substrate and rotatably connected to the first substrate; and a second substrate including first groove portions formed in both ends thereof, the first coupling units being respectively inserted into the first groove portions.

### CROSS-REFERENCE TO RELATED APPLICATIONS

This application claims the priority of Korean Patent Application No. 10-2011-0126048 filed on Nov. 29, 2011, in the Korean Intellectual Property Office, the disclosure of which is incorporated herein by reference.

### BACKGROUND OF THE INVENTION

1. Field of the Invention

The present invention relates to a biochip, and more particularly, to a biochip in which a data chip and a meta chip are precisely coupled to each other.

2. Description of the Related Art

Recently, there has been an increasing need to research and develop biotechnologies for the rapid diagnosis of various human diseases. Accordingly, biochips or cell chips, used for examining biological materials have been steadily developed.

Biochips or cell chips are useful in examining large amounts of biological materials and thus can be used by pharmaceutical companies and cosmetics companies, as well as in hospitals.

According to the related art, a method of examining the reaction of a cell with respect to a particular drug, to investigate the availability and safety (or toxicity) of the particular drug, was used in the pharmaceutical, cosmetics fields, and the like. However, the method according to the related art requires a large amount of reagent in order to perform a precise examination, thereby incurring high costs and requiring a long period of time.

Thus, there is a need to develop biochips for rapidly and accurately performing diagnoses while reducing costs.

Biochips can be classified as deoxyribonucleic acid (DNA) chips, protein chips, and cell chips, according to the type of bio material fixed to a substrate. In early stages of development, as research into human genetic information was emphasized, DNA chips attracted a great deal of attention. However, as interest in proteins as the basis of life processes, and in cells formed by combining proteins as the nuclei of living organisms, has increased, protein chips and cell chips have attracted new interest.

There is a need to develop biochips for obtaining more accurate experimental results at low cost, regardless of the type of biological material analyzed therewith.

In order to obtain accurate examination results, the precise coupling of a data chip, on which a biological cell is cultured, and a meta chip, containing various chemical products or newly developed drugs, is seen as an important step. In particular, since it is difficult to precisely combine the data chip and the meta chip without using automated equipment, there is an acute need to develop a biochip in which the data chip and the meta chip may be combined even manually.

### SUMMARY OF THE INVENTION

An aspect of the present invention provides a biochip in which a data chip and a meta chip are precisely coupled to each other, even through manual handling.

According to an aspect of the present invention, there is provided a biochip, including: a first substrate having a plurality of biological materials disposed thereon at predetermined intervals; first coupling units respectively extending from both ends of the first substrate and rotatably connected to the first substrate; and a second substrate including first groove portions formed in both ends thereof, the first coupling units being respectively inserted into the first groove portions.

According to another aspect of the present invention, there is provided a biochip, including: a first substrate having a plurality of biological materials disposed thereon at predetermined intervals; first coupling units extending from both ends of the first substrate, respectively; and a second substrate including rotatable units that are rotatably installed on both ends thereof, the rotatable units respectively including first groove portions formed therein, and the first coupling units being respectively inserted into the first groove portions.

The biochip may further include first connecting units, each thinner than the first substrate and respectively connecting the first coupling units to both ends of the first substrate, so as to rotate the first coupling units.

Each of the first coupling units may include a flat plate portion connected to each of the first connecting units and a coupling column protruding from the flat plate portion.

The coupling column may have a conical shape, a pyramidal shape, a truncated conical shape, or a truncated pyramidal shape.

The biochip may further include rotatable units respectively including the first groove units formed therein; and second connecting units, each thinner than the second substrate and respectively connecting the rotatable units to both ends of the second substrate, so as to rotate the rotatable units.

The biochip may further include second groove portions formed by engraving one surface of the second substrate; and second coupling units protruding from one surface of the first substrate.

At least one of the first groove portions and the second groove portions may have a rectangular cross-sectional shape.

At least one of the first groove portions and the second groove portions may have a cross-sectional shape that tapers toward a lower portion of the second substrate in a thickness direction thereof.

The first coupling units may respectively contact the first groove portions and then second coupling units may respectively contact the second coupling units so as to couple the first substrate and the second substrate to each other.

The biochip may further include first hinge portions respectively connecting the first coupling units to both ends of the first substrate so as to rotate the first coupling units.

The biochip may further include rotatable units respectively including the first groove portions formed therein; and second hinge portions respectively connecting the rotatable units to both ends of the second substrate, so as to rotate the rotatable units.

The biochip may further include second groove portions formed by engraving one surface of the second substrate; and second coupling units protruding from one surface of the first substrate.

The second substrate may include a plurality of micro wells accommodating the biological materials or drugs.

The first substrate may include protrusions fixing the biological materials or drugs.

The first substrate and the second substrate may each be formed of a plastic material.

The first connecting units and the second connecting units may each be formed of the plastic material.

The first substrate or the second substrate may be formed of a hydrophilic material.

The first substrate or the second substrate may be formed of a hydrophobic material.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view of a first substrate, according to an embodiment of the present invention;

FIG. 2 is a perspective view of a second substrate according to an embodiment of the present invention;

FIGS. 3A and 3B are cross-sectional views for describing a process of coupling the first substrate and the second substrate of FIGS. 1 and 2, according to an embodiment of the present invention;

FIG. 4 is a perspective view of a second substrate, according to another embodiment of the present invention;

FIG. 5 is a cross-sectional view of a case where a first substrate and a second substrate are coupled to each other, according to another embodiment of the present invention;

FIGS. 6A and 6B are cross-sectional views of a case where a first substrate and a second substrate are coupled to each other, according to another embodiment of the present invention;

FIG. 7 is a perspective view of a first substrate, according to another embodiment of the present invention;

FIG. 8 is a perspective view of a second substrate, according to another embodiment of the present invention;

FIGS. 9A and 9B are cross-sectional views for describing a process of coupling the first substrate and the second substrate of FIGS. 7 and 8, according to another embodiment of the present invention;

FIG. 10 is a perspective view of a first substrate, according to another embodiment of the present invention;

FIG. 11 is a perspective view of a second substrate, according to another embodiment of the present invention;

FIGS. 12A and 12B are cross-sectional views for describing a process of coupling the first substrate and the second substrate of FIGS. 10 and 11, according to another embodiment of the present invention; and

FIGS. 13A and 13B are cross-sectional views for describing a process of coupling a first substrate and a second substrate, according to another embodiment of the present invention.

### DETAILED DESCRIPTION OF THE EMBODIMENTS

Embodiments of the present invention will now be described in detail with reference to the accompanying drawings. However, it should be noted that the spirit of the present invention is not limited to the embodiments set forth herein and those skilled in the art and understanding the present invention can easily accomplish retrogressive inventions or other embodiments included in the spirit of the present invention by the addition, modification, and removal of components within the same spirit, but those are construed as being included in the spirit of the present invention.

Further, like reference numerals will be used to designate like components having similar functions throughout the drawings within the scope of the present invention.

For reference, through this specification, a data chip on which a biological cell is cultured may be referred to as a first substrate and a meta chip containing various chemical drugs or newly developed drugs may be referred to as a second substrate. However, according to the biological cell and the chemical drug, functions of the first substrate and the second substrate may be reversed.

In addition, the first substrate and the second substrate according to embodiments of the present invention may each be formed of, for example, silicon, glass, a metal, a polymer, or the like, but are not limited thereto.

Examples of the polymer may include, but are not limited to, polymethylmethacrylate (PMMA), polycarbonate (PC), polystyrene (PS), polypropylene, cyclic olefin copolymer, polynorbonene, styrene-butadiene copolymer (SBC), or acrylonitrile butadiene styrene.

A method of manufacturing the first substrate and the second substrate is not particularly limited. For example, the first substrate and the second substrate may be manufactured by using a photoresist process, an etching process, an injection molding process, or the like.

In addition, the term “biological material” used herein that is attached to or accommodated in the first substrate or the second substrate may refer to various materials including a biological material and is not limited to a biological material itself. For example, arrangements of nucleic acid such as ribonucleic acid (RNA), deoxyribonucleic acid (DNA), or the like, a peptide, a protein, lipid, organic or inorganic chemical molecules, a virus particle, a procaryotic cell, a cell organelle, or the like may be used instead of the biological material. The biological material may not be limited to human cells and may include various animal or vegetable cells.

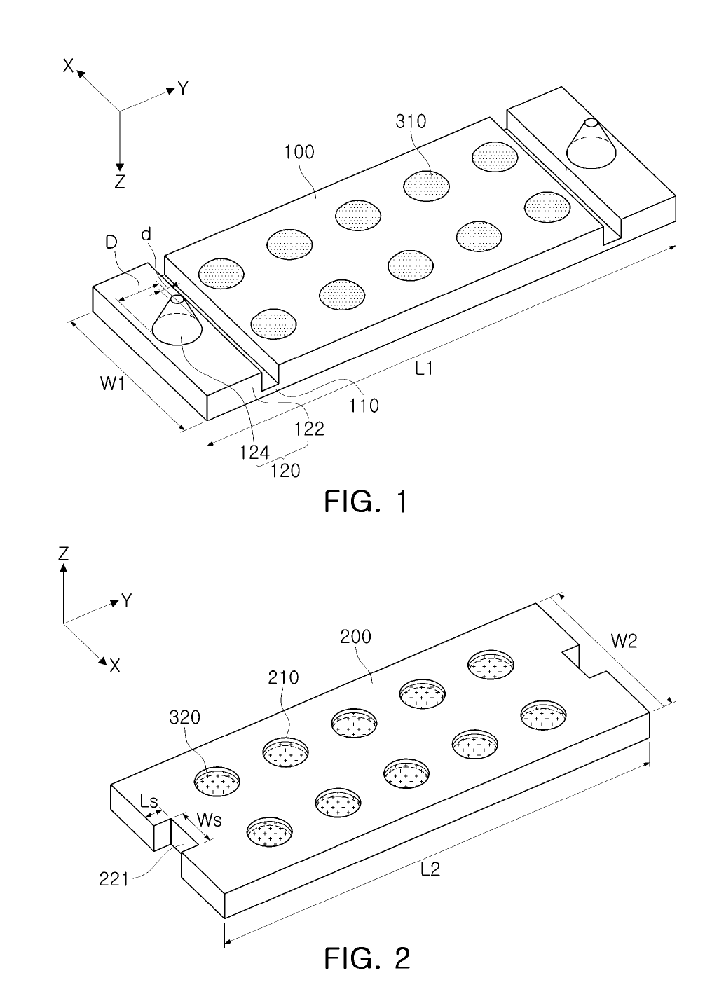

FIG. 1 is a perspective view of a first substrate 100, according to an embodiment of the present invention. FIG. 2 is a perspective view of a second substrate 200 according to an embodiment of the present invention.

Referring to FIGS. 1 and 2, a biochip according to the present embodiment may include the first substrate 100, first coupling units 120, first connecting units 110 for connecting the first coupling units 120 and the first substrate 100, and the second substrate 200.

As shown in FIG. 1, the first substrate 100 may have an approximately thin plate shape.

In detail, the first substrate 100 may have a rectangular shape having a length of L1 and a width of W1. The first substrate 100 having the rectangular shape may be used as a data chip on which a plurality of biological materials 310 are positioned at predetermined intervals.

The first substrate 100 may be formed of a plastic material. However, in consideration of the biological materials 310 or the like attached onto the first substrate 100, the first substrate 100 may be formed of another material. When the first substrate 100 is used as a data chip, the first substrate 100 may be formed of a hydrophilic material so that the biological materials 310 may be easily attached to the first substrate 100.

In detail, a portion of the first substrate 100, to which the biological materials 310 are to be attached, may be coated with a hydrophilic material. A portion of the first substrate 100, to which the biological materials 310 are not to be attached, may be formed of or coated with a hydrophobic material.

The first coupling units 120 may respectively extend from both ends of the first substrate 100 and may be rotatably connected to the first substrate 100.

In detail, the first coupling units 120 may be respectively connected to both ends of the first substrate 100 by the first connecting units 110. The first connecting units 110 may each be thinner than the first substrate 100.

Since the first connecting units 110 are each thinner than the first substrate 100 and are formed of a plastic material, the first connecting units 110 may be bendable. Thus, the first coupling units 120 may be rotatably connected to both ends of the first substrate 100 by the first connecting units 110, respectively.

Each of the first coupling units 120 may include a flat plate portion 122 connected to each of the first connecting units 110 and a coupling column 124 protruding from the flat plate portion 122.

In this case, the coupling column 124 may have a conical shape, a pyramidal shape, a truncated conical shape, or a truncated pyramidal shape.

According to the present embodiment, the coupling column 124 may have a shape of a truncated pyramid having a maximum diameter of D and a minimum diameter of d.

In this case, the maximum diameter D of the coupling column 124 may be greater than a width Ws of a first groove portion 221 formed at one of both ends of the second substrate 200 and may be greater than a length Ls of the first groove portion 221. Under this condition, the coupling column 124 and the first groove portion 221 may point-contact each other. Accordingly, a coupling position of the first substrate 100 and the second substrate 200 may be gradually corrected.

As shown in FIG. 2, the second substrate 200 may have an approximately thin plate shape.

In detail, the second substrate 200 may have a rectangular shape having a length of L2 and a width of W2. The second substrate 200 having the rectangular shape may be used as a meta chip for accommodating drugs 320, reagents, or the like.

The second substrate 200 may include a plurality of micro wells 210 for accommodating the drugs 320, the reagents, or the like. The micro wells 210 may be arranged with a predetermined interval therebetween.

The biological materials 310 and the drugs 320 or the reagents may be respectively positioned on the first substrate 100 and the second substrate 200 with predetermined intervals so as to contact each other when the first substrate 100 and the second substrate 200 are coupled to each other. Thus, a reaction of the biological materials 310 with respect to the drugs 320 or the reagents may be measured.

The second substrate 200 may be formed of a plastic material. Since the second substrate 200 formed of plastic material may be mass-produced by using a mold, manufacturing costs of a biochip including this second substrate 200 may be reduced compared to a biochip formed of glass.

In addition, since the second substrate 200 formed of plastic material is lighter and has lower brittleness than a case of glass material, it may be convenient to handle the biochip including the second substrate 200 formed of a plastic material, thereby reducing damage incidence due to mishandling.

The first groove portions 221 may be respectively formed in both ends of the second substrate 200. Coupling columns 124 of the first coupling units 120 may be respectively inserted into the first groove portions 221 so that the first substrate 100 and the second substrate 200 may be coupled to each other.

The first groove portions 221 may each have a rectangular cross-sectional shape that has a width of Ws and a length of Ls.

However, a shape of each of the first groove portions 221 is not limited to the above-described shape. The first groove portion 221 may have a triangular cross-sectional shape, a pentagonal cross-sectional shape, an octagonal cross-sectional shape, or the like, as well as the rectangular cross-sectional shape.

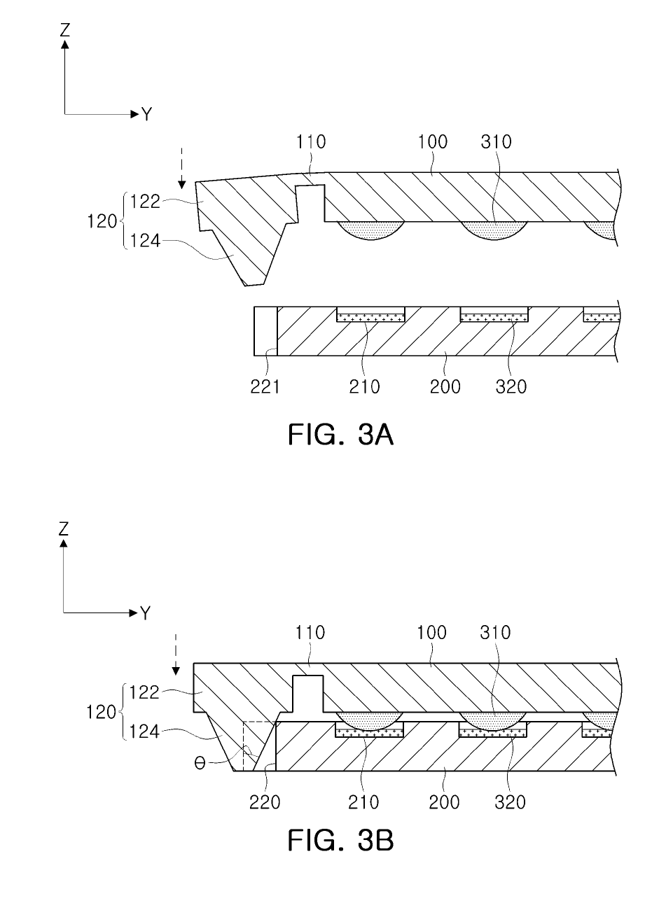

FIGS. 3A and 3B are cross-sectional views for describing a process of coupling the first substrate 100 and the second substrate 200 of FIGS. 1 and 2, according to an embodiment of the present invention.

With reference to FIGS. 3A and 3B, the process of coupling the first substrate 100 and the second substrate 200 will be described.

The first substrate 100 and the second substrate 200 may be coupled by placing the first substrate 100 on the second substrate 200.

That is, while the second substrate 200 may be fixed in a state in which the drugs 320 or the reagents are received in the micro wells 210, the first substrate 100 may be moved from above toward the second substrate 200 so as to be coupled to the second substrate 200.

According to the present embodiment, since the coupling column 124 included in each of the first coupling units 120 has a conical shape having an inclined surface, while the first substrate 100 is moved downward so that a relative position of the first substrate 100 with respect to the second substrate 200 is changed, the first substrate 100 and the second substrate 200 may be aligned.

In an initial state for coupling the first substrate 100 and the second substrate 200, the coupling columns 124 may be respectively inserted into the first groove portions 221 and then the first substrate 100 and the second substrate 200 may be disposed with a predetermined interval therebetween so as not to contact each other.

In this case, since the first connecting units 110 are bendable, the first coupling units 120 may be rotatable by the first connecting units 110. Thus, positions of the first groove portions 221 into which coupling columns 124 included in the first coupling units 120 are respectively inserted may be relatively easily determined. In addition, the coupling columns 124 may be easily inserted into the first groove portions 221, even through manual handling without using automation equipment.

The coupling columns 124 may be respectively inserted into the first groove portions 221 and then the first substrate 100 may be moved downward so that the biological materials 310 positioned on the first substrate 100 may contact the drugs 320 or the reagents positioned on the second substrate 200, thereby completely coupling the first substrate 100 and the second substrate 200 to each other.

While the first substrate 100 is moved along inclined surfaces of the coupling columns 124, a position of the first substrate 100 may be aligned. Thus, by adjusting inclination angles of the inclined surfaces of the coupling columns 124, a coupling position of the first substrate 100 and the second substrate 200 may be precisely adjusted.

In addition, since the first coupling units 120 are rotatably connected to the first substrate 100, the coupling columns 124 included in the first coupling units 120 may be easily inserted into the first groove portions 221, even through manual handling, thereby increasing coupling precision of the first substrate 100 and the second substrate 200.

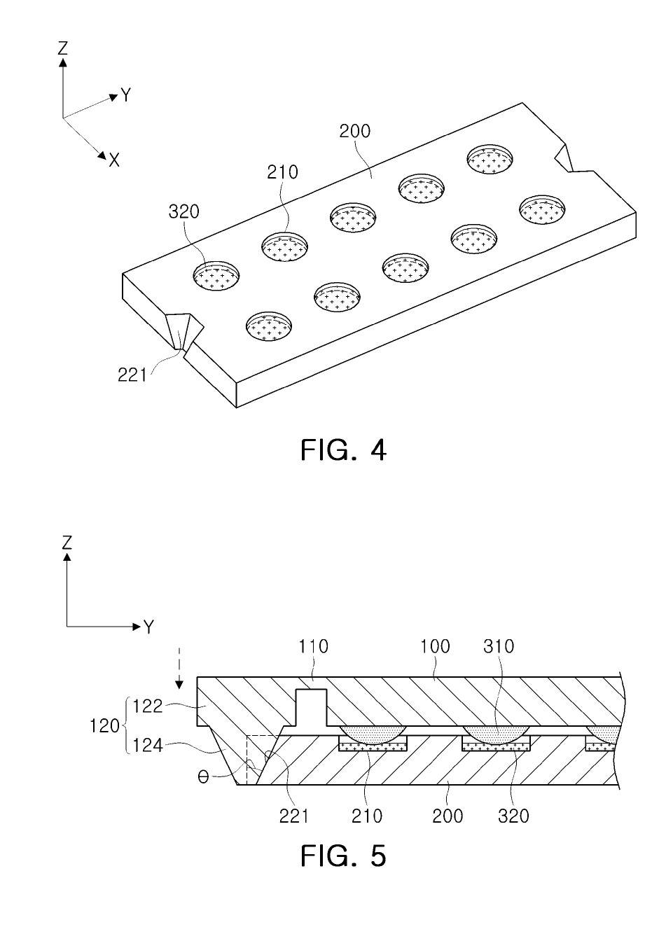

FIG. 4 is a perspective view of a second substrate 200, according to another embodiment of the present invention. FIG. 5 is a cross-sectional view of a case where a first substrate 100 and the second substrate 200 are coupled to each other, according to another embodiment of the present invention.

Referring to FIGS. 4 and 5, the second substrate 200 according to the present embodiment may be different from the second substrate 200 of FIGS. 1 and 2 in terms of the shape of the first groove portion 221.

According to the present embodiment, the first groove portions 221 may each have an inclined surface and may each have a cross-sectional shape that tapers toward a lower portion of the second substrate 200 in a thickness direction of the second substrate 200, as shown in FIGS. 4 and 5.

As shown in FIG. 5, in the biochip configured as described above, the coupling columns 124 and the first groove portions 221 may respectively point-contact each other, and a coupling position of the first substrate 100 and the second substrate 200 may be aligned as the first substrate 100 is moved downward.

Accordingly, the present embodiment may be useful to a case where manufacturing tolerances of the first substrate 100 and the second substrate 200 are relatively high.

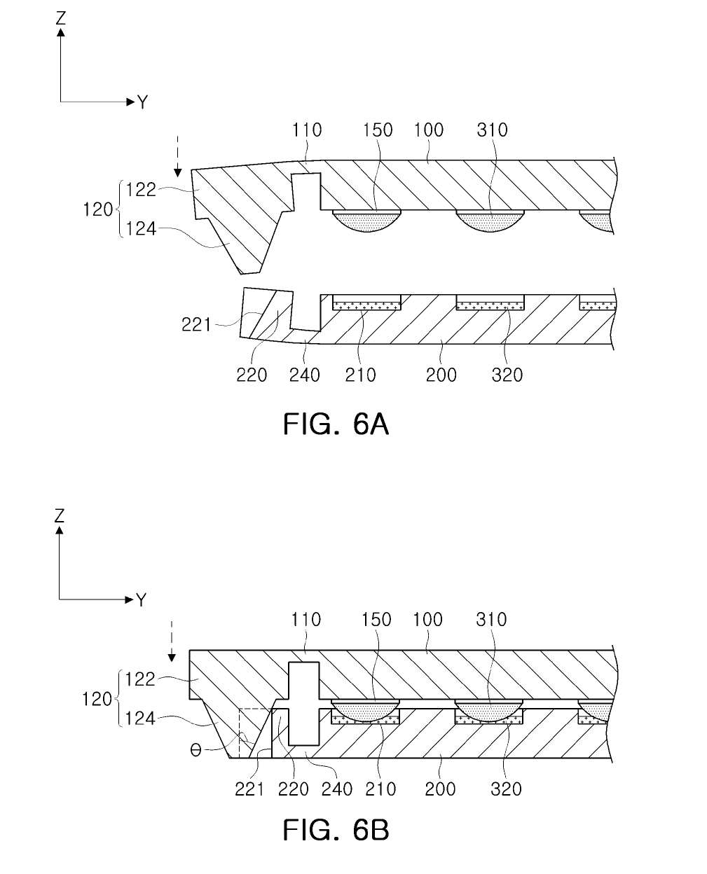

FIGS. 6A and 6B are cross-sectional views of a case where the first substrate 100 and the second substrate 200 are coupled to each other, according to another embodiment of the present invention.

Referring to FIGS. 6A and 6B, the second substrate 200 according to the present embodiment may be different from the second substrate 200 of FIGS. 1 and 2 in that the second substrate 200 according to the present embodiment includes second connecting units 240. In addition, according to the present embodiment, the first substrate 100 may include a plurality of protrusions 150.

According to the present embodiment, rotatable units 220 may be rotatably installed on both ends of the second substrate 200, respectively. The first groove portions 221 may be formed in the rotatable units 220, respectively.

In detail, the rotatable units 220 may be respectively connected to both ends of the second substrate 200 by the second connecting units 240. The second connecting units 240 may each be thinner than the second substrate 200.

Since the second connecting units 240 are each thinner than the second substrate 200 and are formed of a plastic material, the second connecting units 240 may be bendable. Thus, the rotatable units 220 may be rotatably connected to both ends of the second substrate 200 by the second connecting units 240.

According to the present embodiment, since both the first connecting units 110 and the second connecting units 240 are bendable, the first coupling units 120 and the rotatable units 220 including the first groove portions 221 formed therein may be rotatable by the first connecting units 110 and the second connecting units 240, respectively.

By rotating the first coupling units 120 and the rotatable units 220 so as to face each other and respectively inserting the coupling columns 124 included in the first coupling units 120 into the first groove portions 221, although the first substrate 100 and the second substrate 200 are coupled, even through manual handling, coupling failure may be significantly reduced and coupling precision of the first substrate 100 and the second substrate 200 may be relatively increased.

The first substrate 100 may include a plurality of protrusions 150. The protrusions 150 may be formed to correspond to the micro wells 210 formed in the second substrate 200 and may be coated with a hydrophilic material so that the biological materials 310 may be easily attached thereto. In addition, the protrusions 150 may be roughly surface-processed so that the biological materials 310 may be easily attached thereto.

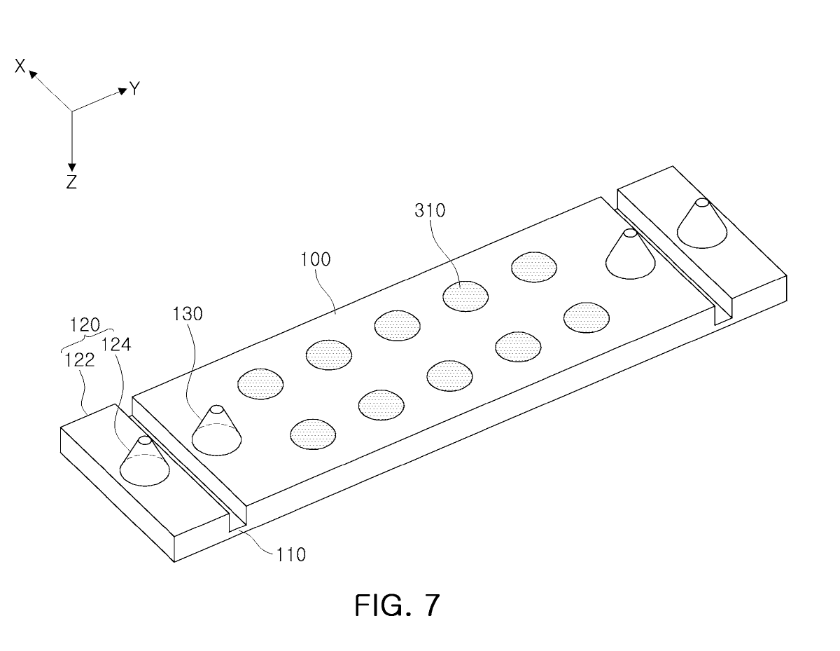

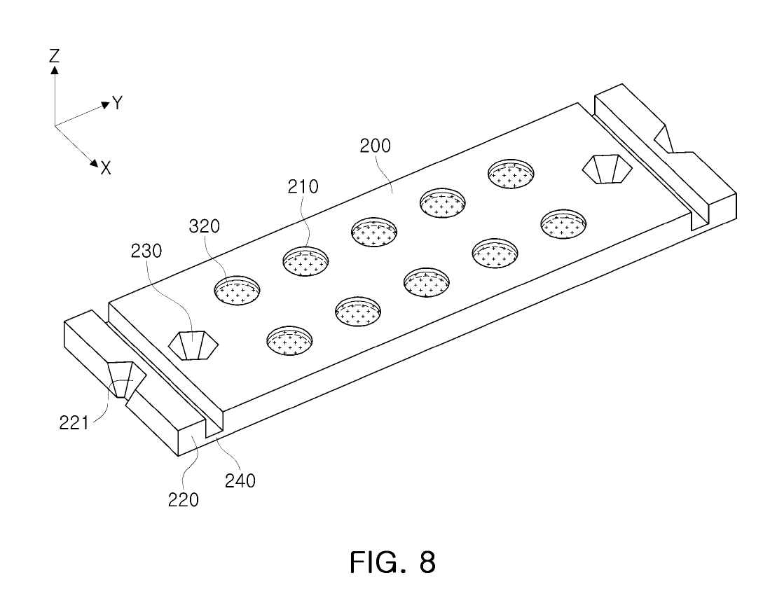

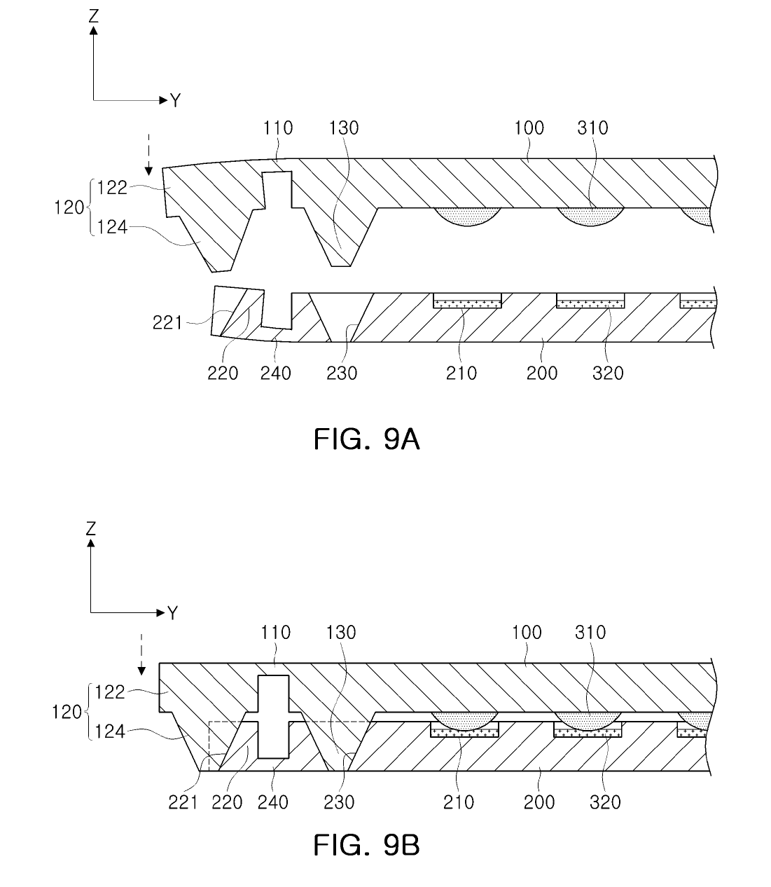

FIG. 7 is a perspective view of a first substrate 100, according to another embodiment of the present invention. FIG. 8 is a perspective view of a second substrate 200, according to another embodiment of the present invention. FIGS. 9A and 9B are cross-sectional views for describing a process of coupling the first substrate 100 and the second substrate 200 of FIGS. 7 and 8, according to another embodiment of the present invention.

Referring to FIGS. 7 through 9A through 9B, the first substrate 100 and the second substrate 200 according to the present embodiment may be different from the first substrate 100 and the second substrate 200 of FIGS. 6A and 6B in that the first substrate 100 and the second substrate 200 according to the present embodiment include second coupling units 130 and second groove portions 230, respectively.

According to the present embodiment, the second groove portions 230 may be formed by engraving one surface of the second substrate 200. The second coupling units 130 may protrude from one surface of the first substrate 100.

In this case, the first substrate 100 and the second substrate 200 may be coupled to each other by respectively inserting the first coupling units 120 into the first groove portions 221, positioning the first substrate 100 and the second substrate 200 with a predetermined interval therebetween so as not to contact each other, and then respectively inserting the second coupling units 130 into the second groove portions 230.

That is, since two coupling processes of the first substrate 100 and the second substrate 200 are sequentially performed, coupling precision of the first substrate 100 and the second substrate 200 may be further improved.

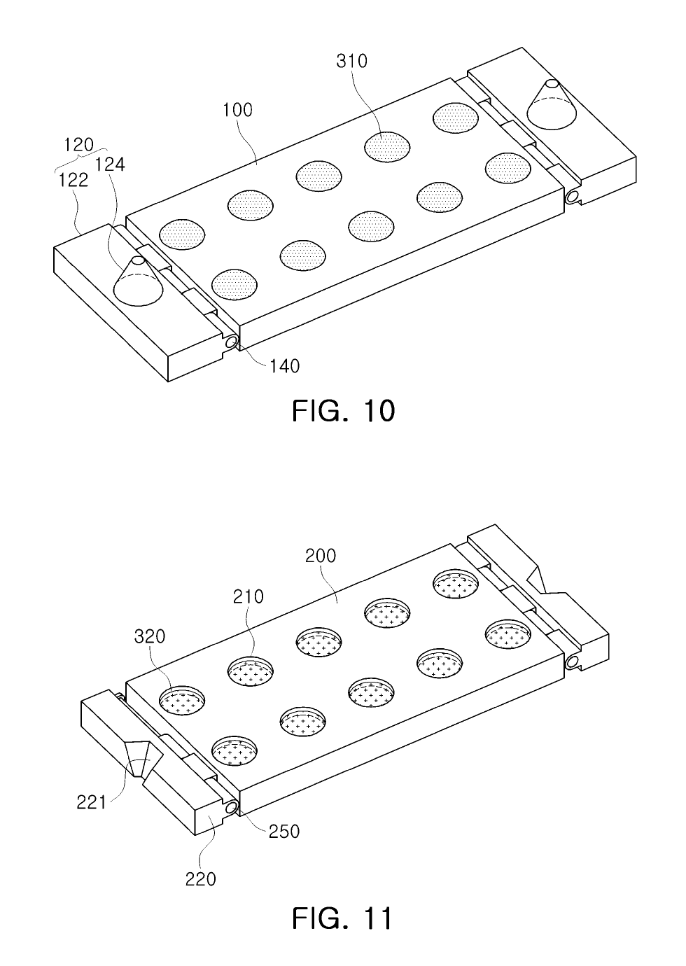

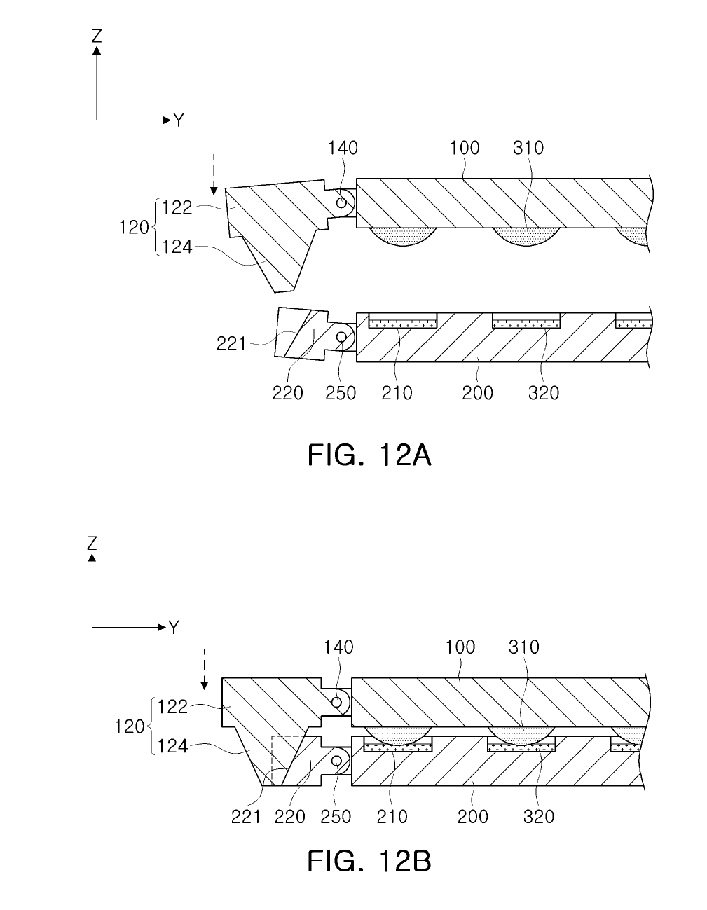

FIG. 10 is a perspective view of a first substrate 100, according to another embodiment of the present invention. FIG. 11 is a perspective view of a second substrate 200, according to another embodiment of the present invention. FIGS. 12A and 12B are cross-sectional views for describing a process of coupling the first substrate 100 and the second substrate 200 of FIGS. 10 and 11, according to another embodiment of the present invention.

Referring to FIGS. 10 through 12A and 12B, the biochip according to the present embodiment may be different from in FIGS. 6A and 6B in that the biochip according to the present embodiment includes first and second hinge portions 140 and 250 so that the first coupling units 120 and the rotatable units 220 including the first groove portions 221 formed therein may be rotatable.

In detail, the first coupling units 120 may be rotatably connected to both ends of the first substrate 100 by the first hinge portions 140, respectively. The rotatable units 220 including the first groove portions 221 formed therein may be rotatably connected to both ends of the second substrate 200 by the second hinge portions 250, respectively.

That is, the first coupling units 120 and the rotatable units 220 may be rotated by the first hinge portions 140 and the second hinge portions 250, respectively. Thus, positions in which the coupling columns 124 included in the first coupling units 120 are inserted into the first groove portions 221 may be more easily determined.

Thus, the coupling columns 124 may be relatively easily inserted into the first groove portions 221, even through manual handling without using automation equipment.

The coupling columns 124 may be inserted into the first groove portions 221 and then the first substrate 100 may be moved downward so that the biological materials 310 positioned on the first substrate 100 and the drugs 320 or the reagents positioned on the second substrate 200 contact each other, thereby completely coupling the first substrate 100 and the second substrate 200 to each other.

According to the present embodiment, both the first coupling units 120 and the rotatable units 220 are rotatable. Alternatively, as needed, only one of the first coupling units 120 and the rotatable units 220 may be rotatable.

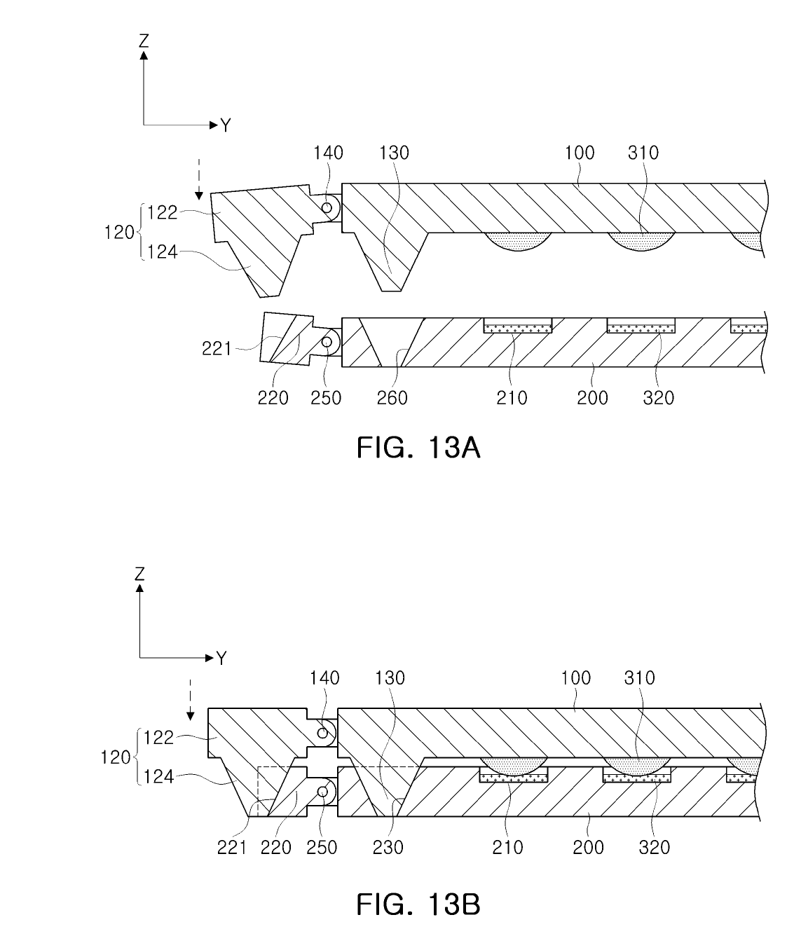

FIGS. 13A and 13B are cross-sectional views for describing a process of coupling a first substrate 100 and a second substrate 200, according to another embodiment of the present invention.

Referring to FIGS. 13A and 13B, the biochip according to the present embodiment may be different from FIGS. 6A and 6B in that the biochip according to the present embodiment includes the first and second hinge portions 140 and 250 so that the first coupling units 120 and the rotatable units 220 including the first groove portions 221 formed therein may be rotatable. In addition, the biochip according to the present embodiment may be different from FIGS. 10 and 11 in that the biochip according to the present embodiment includes the second coupling units 130 and the second groove portions 230.

In detail, the first coupling units 120 may be rotatably connected to both ends of the first substrate 100 by the first hinge portions 140, respectively. The first substrate 100 may include the second coupling units 130 protruding from one surface of the first substrate 100.

In addition, the rotatable units 220 including the first groove portions 221 formed therein may be rotatably connected to the second substrate 200 by the second hinge portions 250, respectively. The second substrate 200 may include the second groove portions 230 formed by engraving one surface of the second substrate 200.

That is, the first coupling units 120 and the rotatable units 220 including the first groove portions 221 formed therein may be rotatable by the first hinge portions 140 and the second hinge portions 250, respectively. The first coupling units 120 may be inserted into the first groove portions 221, the first substrate 100 and the second substrate 200 are positioned with a predetermined interval therebetween so as not to contact each other, and then the second coupling units 130 are respectively inserted into the second groove portions 230. Thus, two coupling processes may be sequentially performed. Due to this structure, although the first substrate 100 and the second substrate 200 are coupled to each other, even through manual handling, coupling failure may be significantly reduced and coupling precision of the first substrate 100 and the second substrate 200 may be increased.

As set forth above, according to the embodiments of the present invention, the biochip may be configured such that the first substrate 100 (or a data chip) and the second substrate 200 (or a meta chip) may be precisely coupled to each other by manual handling without automation equipment, thereby improving the test reliability of the biochip including the first substrate 100 and the second substrate 200.

In addition, in the biochip according to the embodiments of the present invention, since a coupling position of the first substrate 100 and the second substrate 200 may be gradually corrected, the first substrate 100 and the second substrate 200 may be precisely coupled to each other although tolerances are present in terms of shapes of the first substrate 100 and the second substrate 200.

While the present invention has been shown and described in connection with the embodiments, it will be apparent to those skilled in the art that modifications and variations can be made without departing from the spirit and scope of the invention as defined by the appended claims.

What is claimed is:

1. A biochip, comprising:
a first substrate having a plurality of biological materials disposed thereon at predetermined intervals;
first coupling units respectively extending from both ends of the first substrate and rotatably connected to the first substrate; and
a second substrate including first groove portions formed in both ends thereof, the first coupling units being respectively inserted into the first groove portions.

2. A biochip, comprising:
a first substrate having a plurality of biological materials disposed thereon at predetermined intervals;
first coupling units extending from both ends of the first substrate, respectively; and
a second substrate including rotatable units rotatably installed on both ends thereof, the rotatable units respectively including first groove portions formed therein, and the first coupling units being respectively inserted into the first groove portions.

3. The biochip of claim 1, further comprising first connecting units, each thinner than the first substrate and respectively connecting the first coupling units to both ends of the first substrate, so as to rotate the first coupling units.

4. The biochip of claim 3, wherein each of the first coupling units includes a flat plate portion connected to each of the first connecting units and a coupling column protruding from the flat plate portion.

5. The biochip of claim 4, wherein the coupling column has a conical shape, a pyramidal shape, a truncated conical shape, or a truncated pyramidal shape.

6. The biochip of claim 1, further comprising:
rotatable units respectively including the first groove units formed therein; and
second connecting units, each thinner than the second substrate and respectively connecting the rotatable units to both ends of the second substrate, so as to rotate the rotatable units.

7. The biochip of claim 6, further comprising:
second groove portions formed by engraving one surface of the second substrate; and
second coupling units protruding from one surface of the first substrate.

8. The biochip of claim 7, wherein at least one of the first groove portion and the second groove portion has a rectangular cross-sectional shape.

9. The biochip of claim 7, wherein at least one of the first groove portion and the second groove portion has a cross-sectional shape that tapers toward a lower portion of the second substrate in a thickness direction thereof.

10. The biochip of claim 7, wherein the first substrate and the second substrate are coupled to each other by respectively inserting the first coupling units into the first groove portions and then respectively inserting the second coupling units into the second groove portions.

11. The biochip of claim 1, further comprising first hinge portions respectively connecting the first coupling units to both ends of the first substrate, so as to rotate the first coupling units.

12. The biochip of claim 11, further comprising:
rotatable units respectively including the first groove portions formed therein; and
second hinge portions respectively connecting the rotatable units to both ends of the second substrate, so as to rotate the rotatable units.

13. The biochip of claim 12, further comprising:
second groove portions formed by engraving one surface of the second substrate; and
second coupling units protruding from one surface of the first substrate.

14. The biochip of claim 1, wherein the second substrate includes a plurality of micro wells accommodating the biological materials or drugs.

15. The biochip of claim 1, wherein the first substrate includes protrusions fixing the biological materials or drugs.

16. The biochip of claim 1, wherein the first substrate and the second substrate are each formed of a plastic material.

17. The biochip of claim 6, wherein the first connecting units and the second connecting units are each formed of the plastic material.

18. The biochip of claim 1, wherein the first substrate or the second substrate is formed of a hydrophilic material.

19. The biochip of claim 1, wherein the first substrate or the second substrate is formed of a hydrophobic material.
